(19) United States
(12) Patent Application Publication
(10) Pub. No.: US 2026/0174382 A1
(43) Pub. Date: Jun. 25, 2026

(54) KNEE JOINT HEALTH DETECTION DEVICE

(71) Applicant: Yusef Mujtaba, Fort Myers, FL (US)

(72) Inventor: Yusef Mujtaba, Fort Myers, FL (US)

(21) Appl. No.: 18/989,390

(22) Filed: Dec. 20, 2024

Related U.S. Application Data
(60) Provisional application No. 63/617,830, filed on Jan. 5, 2024.

(51) Int. Cl. A61B 5/00; A61B 5/11

(52) U.S. Cl. CPC A61B 5/4585; A61B 5/002; A61B 5/1101; A61B 5/6828; A61B 5/6831; A61B 2560/0443; A61B 2562/0219

(57) ABSTRACT

A wearable device includes a first structure having a first band and a processor mounted on the first band, and a second structure having a second band configured to wrap around a knee and at least one sensor mounted on the second band and configured to measure vibration within the knee. The processor is programmed to make a determination whether the knee is within a range that indicates Osteoarthritis based on the vibration sensed by the at least one sensor, and to output the result to an external device.

### CROSS-REFERENCE TO RELATED APPLICATIONS

This application claims the benefit of U.S. Provisional Application No. 63/617,830 filed Jan. 5, 2024, the disclosure of which is incorporated herein by reference in its entirety.

### INTRODUCTION

The subject disclosure relates to a device for detecting health of a knee joint.

An improved device for detecting health of a knee joint is desirable.

### SUMMARY

In one exemplary embodiment, a wearable device comprises a first structure comprising a first band and a processor mounted on the first band, and a second structure comprising a second band configured to wrap around a knee and at least one sensor mounted on the second band and configured to sense vibration within the knee. The processor is programmed to make a determination whether the knee is within a range that indicates Osteoarthritis based on the vibration sensed by the at least one sensor, and to output the result to an external device.

In addition to one or more of the features described herein, the first band is elastic.

In addition to one or more of the features described herein, the second band is elastic.

In addition to one or more of the features described herein, the first band is configured to wrap around a thigh.

In addition to one or more of the features described herein, the at least one sensor comprises a piezoelectric disk positioned on an interior surface of the second band.

In addition to one or more of the features described herein, the at least one sensor further comprises a piezoelectric accelerometer positioned to a side of the piezoelectric disk on the interior surface of the second band.

In addition to one or more of the features described herein, the at least one sensor further comprises two piezoelectric accelerometers positioned to opposite sides of the piezoelectric disk on the interior surface of the second band.

In addition to one or more of the features described herein, wires connecting the processor and the at least one sensor are bundled together within a protective sleeve.

In addition to one or more of the features described herein, the first structure comprises a pocket disposed on the first band, and the processor is disposed within the pocket.

In addition to one or more of the features described herein, wires connecting the processor and the at least one sensor are bundled together within a protective sleeve, wherein the pocket comprises a wire opening, and wherein the wires extend through the wire opening.

In addition to one or more of the features described herein, the processor is configured to receive sensor data from the at least one sensor and the processor is programmed to process the sensor data.

In addition to one or more of the features described herein, wherein the processor is configured to send results from processing the sensor data to a near-field communication chip.

In addition to one or more of the features described herein, the near-field communication chip is configured to be scanned via an external device for data output on a uniform resource locator.

In addition to one or more of the features described herein, the piezoelectric disk is on the interior surface of the second band at a position that is configured to aligns with a patella of the knee.

In addition to one or more of the features described herein, the piezoelectric disk is on the interior surface of the second band at a position that is configured to aligns with a patella of the knee.

In addition to one or more of the features described herein, the piezoelectric disk is mounted on the interior surface of the second band at a position that is configured to align with a patella of the knee, and the piezoelectric accelerometers are mounted on the interior surface of the second band at positions that are configured to align with femoral condyle of the knee.

In addition to one or more of the features described herein, the wearable device further comprises a first fastener on the first band and a second fastener on the second band.

In addition to one or more of the features described herein, the first fastener and the second fastener are hook and loop fasteners.

In addition to one or more of the features described herein, the processor includes a first threshold above which the vibration indicates a high likelihood of Osteoarthritis, a second threshold under which the vibration indicates a low likelihood of Osteoarthritis.

In addition to one or more of the features described herein, the first threshold is determined based on vibration data gathered when the wearable device is worn by a knee that has Osteoarthritis, and the second threshold is determined based on vibration data gathered when the wearable device is worn by a knee that does not have Osteoarthritis.

The above features and advantages, and other features and advantages of the disclosure are readily apparent from the following detailed description when taken in connection with the accompanying drawings.

### BRIEF DESCRIPTION OF THE DRAWINGS

Other features, advantages and details appear, by way of example only, in the following detailed description, the detailed description referring to the drawings in which:

FIG. 1 is a schematic diagram of a knee joint health detection device according to one or more embodiments;

FIG. 2 is a schematic diagram of a knee joint health detection device according to one or more embodiments;

FIG. 3 shows a knee joint health detection device disposed on a user's leg according to one or more embodiments;

FIG. 4 is a schematic diagram of a piezoelectric disk according to a non-limiting example;

FIG. 5 is a schematic diagram of a piezoelectric accelerometer according to a non-limiting example;

FIG. 6 is a schematic diagram of a portion of a knee joint health detection device according to one or more embodiments; and

FIG. 7 is a schematic diagram of a top portion of a knee joint.

### DETAILED DESCRIPTION

The following description is merely exemplary in nature and is not intended to limit the present disclosure, its application or uses. It should be understood that throughout the drawings, corresponding reference numerals indicate like or corresponding parts and features.

The joints of the human body, especially the synovial knee joints, withstand loads of pressure. These loads lead to damage of the articular cartilage of bones, which may develop into degenerative conditions, limiting the mobility of the body. Osteoarthritis (OA) is the most common type of arthritis that breaks down the cartilage and bones of synovial joints and can lead to atrocious effects if not detected early enough. OA affects the cartilage within a joint which may eventually lead to the breakdown of the underlying bone. If OA is not detected in its early stages, the effects can be devastating as the disease acts rapidly and currently has no cure once fully developed.

The knee is a complex weight-bearing joint and includes the femur, which is at the end of the thigh bone, the tibia, which is at the top of the shin bone, and the patella, which is the knee cap. The end of each of these three bones is covered with a slippery surface, i.e., cartilage, which allows the knee to move smoothly and efficiently. A thickened pad of cartilage called the meniscus is between the thigh bone and the shin bone. The meniscus acts as a shock absorber to cushion the bones and keep the joint stable. The knee joint is wrapped inside a tough capsule filled with synovial fluid. This fluid lubricates and nourishes the cartilage and other structures in the joint.

The symptoms of OA may surface gradually and may include pain in the knee joint, which is often worse after vigorous activity and at the end of the day; pain radiating up into the thigh and/or down into the shin from the affected knee; stiffness of the knee joint, mainly in the morning or after rest, which eases in less than 30 minutes or with walking; swelling of the knee joint which may be soft (caused by additional joint fluid) or hard (caused by bony growths called osteophytes); muscle weakness of the thigh or calf; grinding, creaking or crunching sound when moving the knee; and feeling like the knee “locks”, “sticks”, or gives way during periods of activity. Risk factors for OA include age, weight, gender, and genetic factors.

OA typically starts with damage to the joint cartilage, which is a firm and rubbery tissue that covers the ends of bones and provides a smooth surface for joint movement. This damage can be caused by a variety of factors, including age-related wear and tear, joint injuries or trauma, and repeated stress on the joints from activities such as running or jumping. As the cartilage breaks down, the underlying bone may also undergo changes such as thickening, cyst formation, and the development of bone spurs known as osteophytes. These changes can further contribute to joint damage and inflammation which can cause pain, stiffness, and reduced joint mobility. Inflammation within the joint is a hallmark of OA and can further contribute to joint damage. Inflammatory molecules and enzymes can break down the cartilage and surrounding tissues, leading to further pain and damage. Over time, the joint may become more and more damaged, leading to structural changes such as bone-on-bone contact, joint deformity, and even disability. Additionally, muscle weakness surrounding the affected joint may occur due to disuse or pain, which can further contribute to joint instability and limited mobility. As a result, people with advanced OA may experience joint deformities such as bowing of the legs or swelling of the joints.

There are several current issues with knee OA detection. There is a lack of accurate and reliable diagnostic tools. There is no single diagnostic test for knee OA, and diagnosis is typically based on a combination of clinical examination, medical history, and imaging studies such as X-rays and MRI. However, these tests are not always accurate and may not detect early-stage knee OA. There is also limited accessibility to diagnostic tools. Access to diagnostic tools such as imaging studies can be limited, particularly in low-income countries or remote areas. Knee OA symptoms such as pain and stiffness can be subjective and may vary from person to person, making it difficult to establish a definitive diagnosis. There may also be overreliance on radiographic findings. Radiographic findings such as joint space narrowing and osteophyte formation are commonly used to diagnose knee OA. However, these findings may not correlate well with symptoms, and some people may have radiographic evidence of knee OA without experiencing any symptoms. There may also be a lack of awareness and education among healthcare providers and patients about the importance of early detection of knee OA, leading to delayed diagnosis and management. Currently available treatment options for knee OA focus on symptom management rather than disease modification, and there is a need for more effective therapies to prevent disease progression. Addressing these issues will require a multidisciplinary approach involving healthcare providers, researchers, policymakers, and patients to improve knee OA detection, increase access to diagnostic tools, and develop more effective treatments.

Current methods that detect OA and determine joint health may have negative effects including being invasive, costly, and damaging to the body.

As OA develops and as joint health declines, the synovial fluid starts to become less present, articular cartilage starts to disappear, and the bone degrades over time. As a result, there is a greater amount of friction present between the femur and the tibia. This leads to a greater vibration frequency emitted as the rough ends of the bone rub against each other.

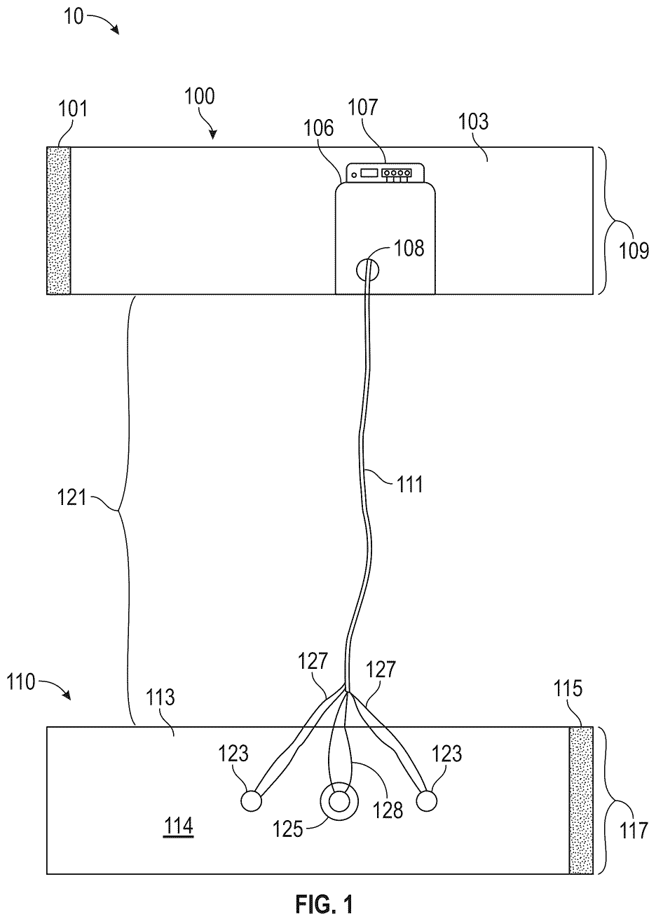

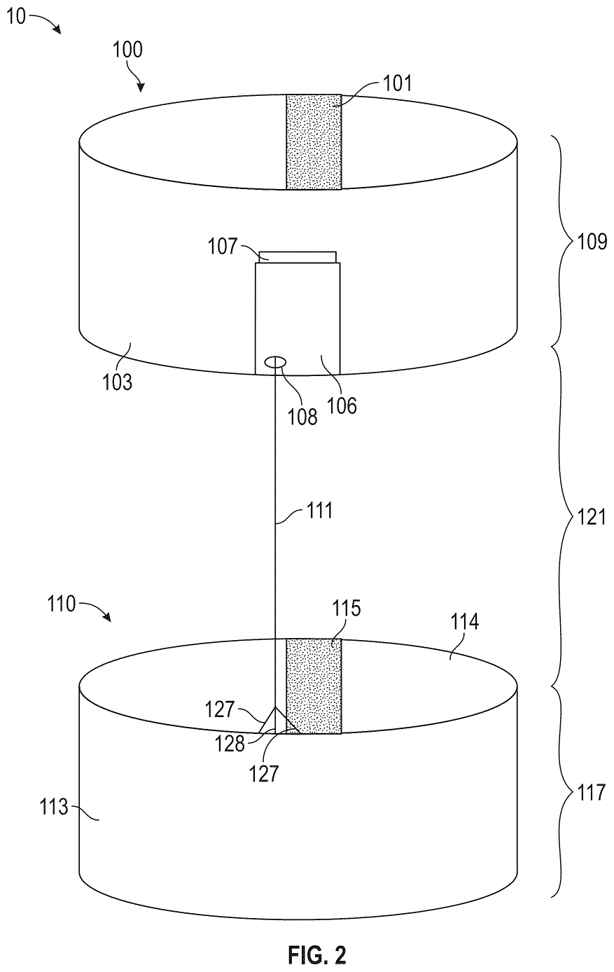

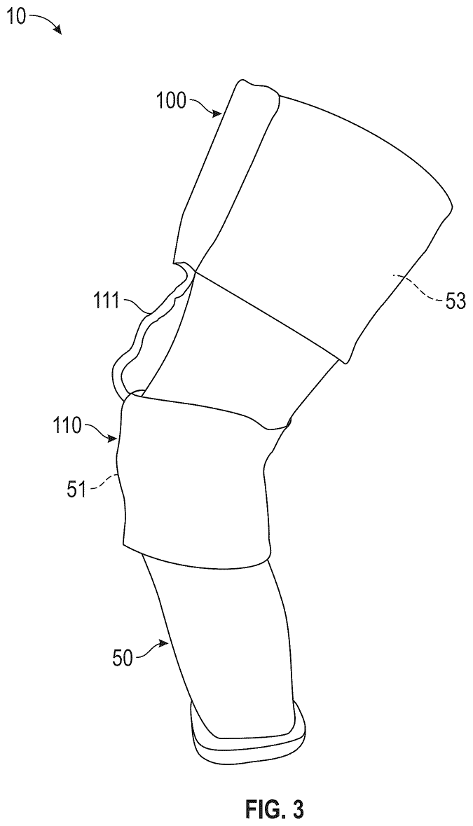

FIG. 1 shows an embodiment of a knee joint health detection device 10 in an unwrapped configuration FIG. 2 shows the knee joint health detection device 10 in a wrapped configuration, and FIG. 3 shows the knee joint health detection device 10 wrapped around a user's leg 50. The knee joint health detection device 10 includes a first structure 100 and a second structure 110. The first structure 100 includes a first band 103 with a first fastener 101 on one end thereof. The first band 103 may be an elastic band, and the first fastener 101 may be a hook and loop fastener. A pocket 106 may be formed in the first band 103, and a wire opening 108 may be formed in the pocket 106. The second structure 110 includes a second band 113 with a second fastener 115 on one end thereof. The second band 113 may be an elastic band, and the second fastener 115 may be a hook and loop fastener. As shown in FIG. 3, the first structure 100 may be disposed around a thigh 53 of a leg 50 of the user, while the second structure 110 may be disposed around a knee 51 of the leg 50 of the user.

The first structure 100 and the second structure 110 may be separated by a gap 121. The first band 103 has a first height 109 and the second band 113 has a second height 117. According to a non-limiting example, the first height 109 may be between 4-8 inches. According to a non-limiting example, the first height 109 may be 6 inches. According to a non-limiting example, the second height 117 may be between 4-8 inches. According to a non-limiting example, the second height 117 may be 6 inches. According to a non-limiting example, the gap 121 may be between 15-25 inches. According to a non-limiting example, the gap 121 may be between 18-22 inches. According to a non-limiting example, the gap 121 may be 20 inches.

A first processor 107 may be disposed within the pocket 106. The first processor 107 may be secured within the pocket 106 such that the first processor 107 does not fall out during use. The first processor 107 may be removable from the pocket 106 such that the first band 103 may be washed without the first processor 107 in the pocket 106.

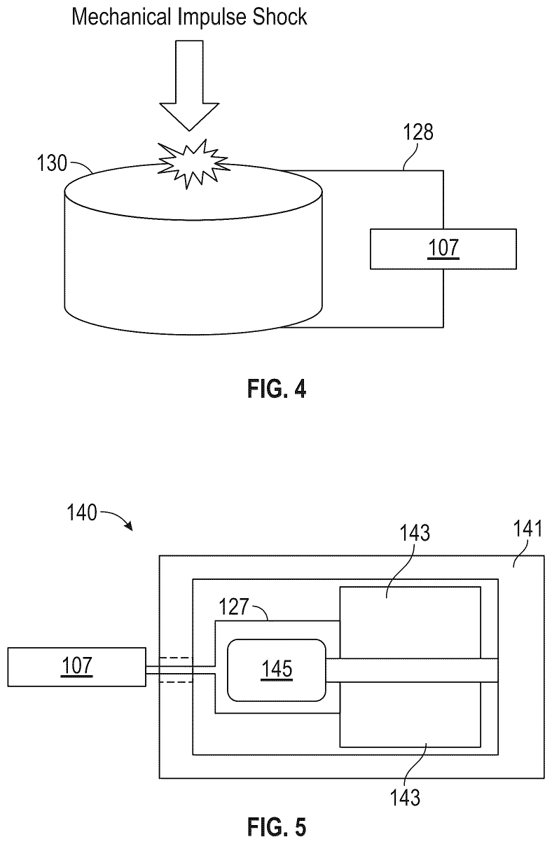

First sensors 123 and a second sensor 125 may be disposed on an interior surface 114 of the second band 113. The first sensors 123 may be piezoelectric (“PZT”) accelerometers 140, a non-limiting example of which is shown in FIG. 5, and the second sensor 125 may be a PZT disk 130, a non-limiting example of which is shown in FIG. 4.

A bundled wire 111 operably connected to the first processor 107 may extend from the first processor 107 through the wire opening 108. The bundled wire 111 may extend across at least a portion of the gap 121 and may include first sub-wires 127 and second sub-wires 128. The bundled wire 111 may include a protective sleeve around the first and second sub-wires 127, 128. The first sub-wires 127 and second sub-wires 128 may split out of the bundled wire 111 proximate to the first and second sensors 123, 125. The first sub-wires 127 may be coupled to the first sensors 123, and the second sub-wires 128 may be coupled to the second sensor 125.

The term PZT refers to the material used in the sensor which is made up of crystals that generate a voltage when subjected to mechanical stress. A PZT disk 130 is a type of sensor that may generate different voltages based on varying vibrational pressure. The PZT disk 130 may be coupled to the second sub-wires 128 and thereby connected to the first processor 107. Thus, by measuring voltage across the PZT disk 130 via the first processor 107, vibrations and frequency within structural objects on which the PZT disk 130 is mounted may be measured.

A PZT accelerometer 140 is a type of sensor that may measure vibration, acceleration, and shock in a variety of mechanical systems. The PZT accelerometer 140 may include a base 141, and PZT elements 143 that are coupled to a weight 145. When the PZT accelerometer 140 is subjected to vibration or acceleration, the crystals inside the PZT elements 143 are compressed or stretched which causes a voltage to be generated across the PZT elements 143. The voltage across the PZT elements 143 is proportional to the acceleration or vibration. The PZT elements 143 may be coupled to the first sub-wires 127 and thereby connected to the first processor 107. By measuring the voltage across the PZT elements 143, the acceleration or vibration may be measured, thereby analyzing the behavior of structural objects on which the PZT accelerometer 140 is mounted. PZT accelerometers 140 are typically small, rugged, and reliable, and can provide accurate measurements over a wide range of frequencies and acceleration levels.

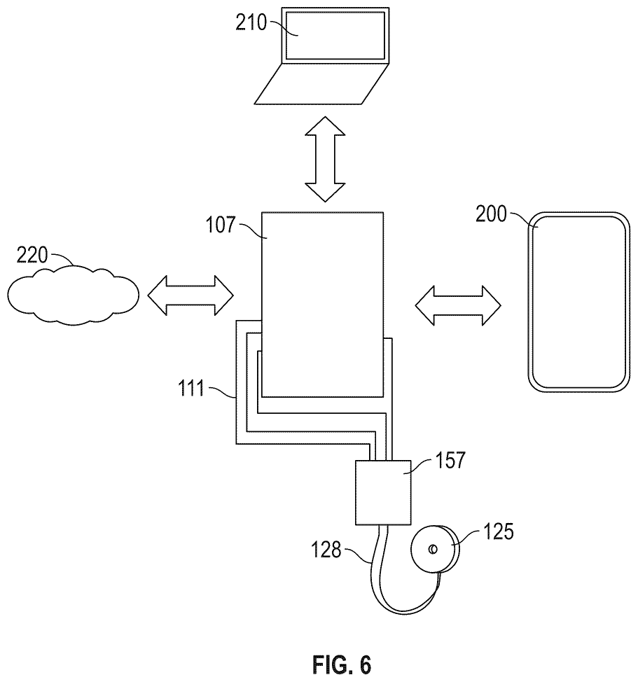

As shown in FIG. 6, a second processor 157 may be coupled to the first processor 107 one or more of the sensors. While FIG. 6 shows the second processor 157 being coupled to the second sensor 125 via the second sub-wires 128, the second processor 157 may be alternatively or additionally coupled to the first sensor(s) 123 via the first sub-wires 127.

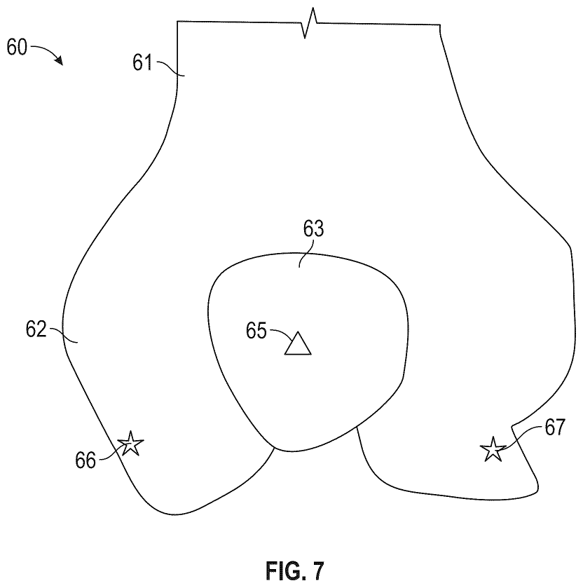

FIG. 7 shows a top portion of a knee joint 60. The knee joint 60 includes a bottom portion of the femur 61 including the femoral condyle 62 and the patella 63. The inventor determined that vibrations for detecting OA should be measured at first position 65 located on the patella 63 and second and third positions 66, 67 located on femoral condyle 62. According to one or more embodiments, the second structure 110 may be configured such that, when the second structure 110 is worn by the user, the second sensor 125 may be positioned so as to align with the first position 65 and the first sensors 123 may positioned so as to align with the second and third positions 66, 67. This alignment may allow for the greatest vibration frequency to be measured within the knee.

A knee joint health detection device 10 according to one or more embodiments may detect and measure change in friction, e.g., change in vibration frequency, via the first sensors 123 and the second sensor 125 and determine stages of joint health and OA detection based on the measured vibration frequency. The results may then be outputted to the user. For example, as shown in FIG. 6, the results may be wirelessly outputted to an external device of the user, e.g., a cellular phone 200, a computer 210, the cloud 220, etc.

A knee joint health detection device 10 according to one or more embodiments may sense vibrations emitted by friction within the knee to determine joint health and detect the possibility of OA. According to a non-limiting example, the knee joint health detection device 10 may include a band 113 in the form of a surrounding knee brace that utilizes first and second sensors 123, 125 in the form of two PZT accelerometers 140 and a PZT disk 130. The first and second sensors 123, 125 may be placed strategically so that measurements of the vibration emitted from the bending of the knee joint 60 may allow for the most efficient capture of friction emitted between the femur 61 and the tibia (not shown). As the user flexes and extends the knee joint 60, the first and second sensors 123, 125 may measure the vibration emitted. The data from the first and second sensors 123, 125 may be transferred using a first processor 107 programmed to output the measurements onto an external device, e.g., a cellular phone 200, a computer 210, the cloud 220, etc. A dataset of measurements from the knee joint health detection device 10 measuring healthy knees and knees of those suffering from OA may be analyzed to generate thresholds.

As a non-limiting example, a first threshold may be set based on the dataset, and if vibration measured by the first and second sensors 123, 125 of the knee joint health detection device 10 is at or exceeds the first threshold, a determination may be made of a high likelihood of OA. As a non-limiting example, a second threshold may be set based on the dataset, and if vibration measured by the sensors of the knee joint health detection device is at or below the second threshold, a determination may be made of a low likelihood of OA. As a non-limiting example, if vibration measured by the knee joint health detection device is between the first threshold and the second threshold, a determination may be made that there is a medium likelihood of OA. One or more embodiments of the present disclosure may determine joint and health and detect OA non-invasively, cost-effectively, and/or without damaging the user.

As a non-limiting example, machine learning may be applied using a dataset of measurements from the knee joint health detection device 10 measuring healthy knees and knees of those suffering from OA. That is, via machine learning, the first processor 107 may correlate vibrations from the sensors to likelihood of OA.

The first structure 100 may wrap around the thigh 53 and receive and compute data from the first and second sensors 123, 125 to determine the presence of OA. The data may be received from the first and second sensors 123, 125 and may include data sensing vibrations when a knee 51 that the input system is disposed on is bent. For example, bending of the knee 51 may be monitored in a predetermined, e.g., one minute, bend test. Vibrations generated due to friction between the degenerative articular cartilages may present anomalous patterns in amplitude and frequency scales when compared with healthy knees, indicating the possibility of OA.

One or more embodiments include a plurality of sensors in the form of first and second sensors 123,25. As a non-limiting example, the input system may include a PZT disk 130 and two PZT accelerometers 140, one on each side of the PZT disk 130. The vibrations measured from the first and second sensors 123, 125 may be analyzed using any statistical method known in the art. For example, the vibration measurements may be analyzed by calculating the total, the mean, median, a weighted average, or each vibration may be analyzed separately.

The knee joint health detection device 10 according to one or more embodiments may include an input system in the form of the second structure 110 and a computational system on the form of the first structure 100.

According to a non-limiting example, the first band 103 and/or the second band 113 may be 6 inches wide by 20 inches long. As non-liming examples, the first and second bands 103, 113 of the first and second may the same size or be different sizes. For example, the first band 103 may be larger than the second band 113. According to one or more embodiments, a near-field communication chip disposed on the first processor 107 may output the data to the user. For example, whether OA is present may be communicated to the user.

As explained herein, first and second fasteners 101, 115, which may be hook and loop fasteners, may be used to secure the first and second bands 103, 113 around the knee 51 or the thigh 53. For example, a certain length of the first and/or second fastener 101, 115, e.g., six inches thereof, may be attached to ends of the first and/or second band 103, 113 via, e.g., an adhesive or other attachment structures.

As explained herein, a pocket 106 may be formed on the first band 103 of first structure 100. The pocket 106 may hold a first processor 107 which may include a microprocessor, boards, and/or other electrical components. The pocket 106 may be sewn onto the first band 103, formed integrally therewith, or may be formed using other methods/structures known in the art. The first structure may include one or more processors, e.g., first and second processors 107, 157. The first and/or second processors 107, 157 may include a near-field communication chip.

The first and second sensors 123, 125 may be attached within the second structure. For example, foam tape may attach the first and second sensors 123, 125 to the second band 113. The first and second sensors 123, 125 may be removably attached to the second band 113 so that the second band 113 may be washed without the first and second sensors 123, 125.

A website or an application may display the results of the vibration sensors through scanning the near-field communication chip. The first structure 100 may determine whether OA is present or not, and the extent of the OA if present, and output the result to an external device, e.g., a cellular phone 200, a computer 210, the cloud 220, etc.

The first structure 100 is an example of a computational system and the second structure 110 is an example of an input system.

The terms “a” and “an” do not denote a limitation of quantity, but rather denote the presence of at least one of the referenced item. The term “or” means “and/or” unless clearly indicated otherwise by context. Reference throughout the specification to “an aspect”, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

When an element such as a layer, film, region, or substrate is referred to as being “on” another element, it can be directly on the other element or intervening elements may also be present. In contrast, when an element is referred to as being “directly on” another element, there are no intervening elements present.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this disclosure belongs.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof.

What is claimed is:

1. A wearable device comprising:
a first structure comprising a first band and a processor mounted on the first band; and
a second structure comprising a second band configured to wrap around a knee and at least one sensor mounted on the second band and configured to sense a vibration within the knee;
wherein the processor is programmed to make a determination whether the knee is within a range that indicates Osteoarthritis based on the vibration sensed by the at least one sensor, and to output a result to an external device.

2. The wearable device of claim 1, wherein the first band is elastic.

3. The wearable device of claim 1, wherein the second band is elastic.

4. The wearable device of claim 1, wherein the first band is configured to wrap around a thigh.

5. The wearable device of claim 1, wherein the at least one sensor comprises a piezoelectric disk positioned on an interior surface of the second band.

6. The wearable device of claim 5, wherein the at least one sensor further comprises a piezoelectric accelerometer positioned to a side of the piezoelectric disk on the interior surface of the second band.

7. The wearable device of claim 5, wherein the at least one sensor further comprises two piezoelectric accelerometers positioned to opposite sides of the piezoelectric disk on the interior surface of the second band.

8. The wearable device of claim 1, wherein wires connecting the processor and the at least one sensor are bundled together within a protective sleeve.

9. The wearable device of claim 1, wherein the first structure comprises a pocket disposed on the first band, and the processor is disposed within the pocket.

10. The wearable device of claim 9, wherein wires connecting the processor and the at least one sensor are bundled together within a protective sleeve, wherein the pocket comprises a wire opening, and wherein the wires extend through the wire opening.

11. The wearable device of claim 8, wherein the processor is configured to receive sensor data from the at least one sensor and the processor is programmed to process the sensor data.

12. The wearable device of claim 11, wherein the processor is configured to send results from processing the sensor data to a near-field communication chip.

13. The wearable device of claim 12, wherein the near-field communication chip is configured to be scanned via an external device for data output on a uniform resource locator.

14. The wearable device of claim 5, wherein the piezoelectric disk is on the interior surface of the second band at a position that is configured to aligns with a patella of the knee.

15. The wearable device of claim 6, wherein the piezoelectric disk is mounted on the interior surface of the second band at a position that is configured to align with a patella of the knee, and the piezoelectric accelerometer is mounted on the interior surface of the second band at a position that is configured to align with femoral condyle of the knee.

16. The wearable device of claim 7, wherein the piezoelectric disk is mounted on the interior surface of the second band at a position that is configured to align with a patella of the knee, and the piezoelectric accelerometers are mounted on the interior surface of the second band at positions that are configured to align with femoral condyle of the knee.

17. The wearable device of claim 1, further comprising a first fastener on the first band and a second fastener on the second band.

18. The wearable device of claim 17, wherein the first fastener and the second fastener are hook and loop fasteners.

19. The wearable device of claim 1, wherein the processor includes a first threshold above which the vibration indicates a high likelihood of Osteoarthritis, a second threshold under which the vibration indicates a low likelihood of Osteoarthritis.

20. The wearable device of claim 19, wherein the first threshold is determined based on vibration data gathered when the wearable device is worn by a knee that has Osteoarthritis, and the second threshold is determined based on vibration data gathered when the wearable device is worn by a knee that does not have Osteoarthritis.
